(19) United States
(12) Patent Application Publication
(10) Pub. No.: US 2016/0051354 A1
(43) Pub. Date: Feb. 25, 2016

(54) SLING SYSTEM WITH REMOVABLE STRING

(71) Applicants: Mangesh PATANKAR, Navi Mumbai (IN); Dinesh DIWAKAR, Mumbai (IN); Sandeep AMBARDEKAR, Mumbai (IN)

(72) Inventors: Mangesh PATANKAR, Navi Mumbai (IN); Dinesh DIWAKAR, Mumbai (IN); Sandeep AMBARDEKAR, Mumbai (IN)

(21) Appl. No.: 14/783,389

(22) PCT Filed: Apr. 16, 2014
(86) PCT No.: PCT/IN2014/000238

§ 371 Date: Oct. 8, 2015

(30) Foreign Application Priority Data
May 2, 2013 (IN) 1593/MUM/2013

(51) Int. Cl. A61F 2/00

(52) U.S. Cl. CPC A61F 2/0036

(57) ABSTRACT

An efficient, safe, and reproducible surgical technique for the treatment of female stress urinary incontinence, in which the anchor consists of removable string so that no string remains inside the body after positioning, fixation and adjustment of the sling in the target place during surgery and thus minimizing the subjects discomfort after placement of the sling. Thus allowing removal of the string from the passage of the anchor for short sling system. The short and long sling system further comprises fixation of the bearing, ring and the attachment that allows for anti-twisting element to provide better flexible attachment of the sling with the delivery device facilitating relative rotational motion of the bearing with the ring and the attachment but preventing axial displacement of the bearing with the attachment. [0035] The invention further contains a device which allows attachment of the sling with the delivery device by threading/screwing during assembly. This invention relates to a method of placing the sling in a position beneath the urethra by transobturator approach using small incision through the marked position at the vaginal epithelium, beneath the level of mid-urethra, as per standard procedure for a midurethral sling for inside-out approach and making a thigh incision at the marked position, placing the sling beneath the mid-urethra through transobturator approach outside-in approach. This procedure involves for both long sling system and short sling system.

### FIELD OF THE INVENTION

The present invention relates generally to the field of medical devices for treating stress urinary incontinence in a human or animal subject and in particular to a short sling system with anchor that allows for removable string and a sling fixation of the bearing, attachment and the ring that contains anti-twisting feature for short and long sling system. The invention also allows for inside-out approach and outside-in approach for long sling and short sling system.

### BACKGROUND OF THE INVENTION

Stress urinary incontinence (“SUI”) is a widespread problem throughout the world affecting people of all ages and gender. SUI is the involuntary leakage of small amounts of urine resulting from an increased pressure in the abdomen and may result while sneezing, coughing, laughing, bending, lifting, etc. While primarily a female problem, men also suffer from stress urinary incontinence, and rates of male SUI are increasing along with the increased use of prostate surgery. Stress incontinence in men is typically the result of a weakened urethral sphincter that surrounds the prostate, frequently as a result of prostate surgery.

For treating SUI, it is often necessary to resort to surgery. Conventional techniques consist of restoring the natural mechanisms of continence, maintaining the urethra in the abdominal cavity, and/or increasing urethral resistance. To do this, a conventional sling is placed under the urethra, thereby making it possible to improve the suspension and provide some compression of the urethra. Currently, there are a variety of different sling procedures which differ in the anchoring methods and materials used.

Despite advances in mid-urethral sling design over the past years, there still remains considerable room for improvements, particularly in sling design and placement. For example, such procedures typically require hospitalization. Thus, many females and males with stress urinary incontinence avoid or delay undergoing an operation. Moreover, although serious complications associated with sling procedures are infrequent, they do occur. In some cases, the slings cause friction in the area of the vagina or urethra during the patient's movements and may injure different organs with which they are in contact. This friction may then cause erosion, inflammation or infection, or even cause rejection of the sling, thereby requiring another operation to surgically remove the sling.

Other shortcomings of known sling designs include the fact that multiple incisions are typically required to implant a sling, thereby increasing the patient's level of discomfort and recovery time. Additionally, passage of mesh through the skin or subcutaneous tissue can result in patient discomfort and therefore most commonly requires general anesthesia. Moreover, once implanted, the sling cannot be adjusted, and thus if the sling is not implanted in the precise or ideal location, the patient may continue to have incontinence-related issues. There are some devices whose use compromise the surgeon's ability to easily and accurately tension the sling. This is being perceived as a major shortcoming, and is probably a major reason underlying unacceptable initial failure rates of approximately 25%.

Accordingly, there exists a need for a sling that satisfactorily treats stress urinary incontinence and that permits post-operative adjustment of the sling. Further needs exist for methods for implanting slings that minimize a subject's discomfort and recovery time and allow for placement under local anesthesia.

### SUMMARY OF THE INVENTION

The invention overcomes the deficiencies of the prior arts and provides for a surgical device and method for the treatment of female stress urinary incontinence. This invention relates to a device and method of placement of the sling in a position beneath the urethra by transobturator approach using small incision through the marked position at the vaginal epithelium, beneath the level of mid-urethra, as per standard procedure for a midurethral sling for short sling inside-out approach and long sling inside-out approach as well as short sling outside-in approach and short sling outside-in approach.

The procedure for inside-out approach for short sling system using right and left hand hemispherical delivery device involves connecting the attachment of one end of the sling to the passer end by threading of the delivery device. Rotating the threading attachment for three times. Then placing the passer end at the vaginal incision and pushing the passer through the tissue so that the passer tip passes the obturator foramen. The anchor will allow insertion and proper positioning of the anchor into the tissues of the obturator foramen that will prevent displacement of the sling from the position beneath the urethra.

When the attachment along with some portion of string appears at the skin opening, grasp it with a forcep and pull both the free ends of the string from both sides to position the mesh. The string is pulled until the anchor on the mesh end gets fixed to the tissues in the obturator foramen on both the sides. The exposed string strips are cut and pulled to remove the entire string from the body.

The procedure for short sling system outside-in approach using right and left hand helical delivery device involves attaching the passer threading of the delivery device to the sling system, making a thigh incision at the marked position, allowing the point of the handle to exit near the previously determined exit point at the vaginal epithelium beneath the level of mid-urethra, connecting the attachment on one end of the string to the passer threading of the delivery device, rotating the threading, gently applying traction on the handle to draw the passer back through the thigh incision, till the string can be held by forceps, repeating the procedure on the other side of the patient, allowing the anchor to tension in the tissues of the obturator foramen within the pubic bones, pulling the string until the anchor on the mesh end gets fixed to obturator muscles, placing the sling beneath the mid-urethra through transobturator approach, the anti-twist element prevents twisting of the mesh, pulling and retracting the entire string system from the distal end of the anchor, cutting the exposed string strips and pulling and retracting it to remove the entire strip from the body.

The procedure for long sling system inside-out approach using right and left hand hemispherical delivery device involves attaching the passer threading of the delivery device to the sling system, making a vaginal incision, placing the sling beneath the mid-urethra through transobturator approach, allowing the anchor to tension in the tissues of the obturator foramen within the pubic bones, the anti-twist element prevents twisting of the mesh, pulling the free ends of the mesh, cutting the attachment on the mesh ends, pulling and positioning the free ends of the mesh, pulling and retracting the entire sheath from the outer edges of the mesh, cutting the exposed mesh strips and pulling it to remove the exposed strip from the body.

The procedure for long sling outside-in approach using right and left hand helical delivery device involves making an incision at one of the previously marked position at the level of clitoris in the folds of the thigh. When the complete passer thread along with its straight edge appears at the skin opening, the attachment on one end of the sling is connected to the passer end by threading of the delivery device. Then the threading attachment is rotated for three times. In this technique also the anchor will allow insertion and proper positioning of the anchor into the tissues of the obturator membrane that will prevent displacement of the sling from the position beneath the urethra, gently applying traction on the handle to draw the passer back through the thigh incision, till the sling can be held by forceps, repeating the procedure on the other side of the patient, allowing the anchor to tension in the tissues of the obturator foramen within the pubic bones, cutting the attachment on the mesh ends, pulling and positioning the free ends of the mesh, pulling and retracting the entire sheath from the outer edges of the mesh, cutting the exposed mesh strips and pulling it to remove the exposed strip from the body.

For this purpose, the sling has been designed in a way so as to adjust the sling in a manner such that the sling is in a proper placing with respect to the urethra for providing proper support to the urethra and hence deal with the problem of stress urinary incontinence. The objective of the invention comprises of a method of placing the sling such that after the procedure of insertion of the sling is performed the free end of the string is pulled from both the sides from the passage of the anchor until the fins of the anchor gets fixed on the obturator foramen and strings from both the sides can be removed from the sling and hence no string remains inside the body thus eliminating discomfort to the patient. This also prevents string erosion inside the body.

The objective of the invention is a sling that will be properly positioned into the patient's body without any special instrument, just by using a standard surgical forceps (Right hand hemispherical and left hand hemispherical, Right hand helical and Left hand helical) especially designed for inside-out and outside in approach. An advantage of the invention is that it is useful across different medical specialties depending on preferred surgical approaches.

The sling device has attachment on the sling system which consists of anti-twisting element on both the sides such that when the sling system is assembled there is relative rotational motion of the attachment and the ring with respect to the bearing but no axial displacement or dislodgment of the bearing with respect to the attachment that prevents anti-twisting movement. The anti-twist element will prevent twisting of the sling system device later after the procedure is completed or when the insertion is carried on and during assembling of the passer/handle with the sling. The anti-twisting element will allow relative rotational motion of the attachment and the ring with the bearing of the sling system assembly but will prevent axial displacement or dislodging of the attachment with the bearing.

The invention also discloses a feature where exposed string strips allow removal of the string from the passage of the slots in the anchor by pulling the free end of the string to remove the entire string from the body, the string from side is pulled and string from side is retracted from the anchor as shown in FIG. 17. The string is pulled through the axial hole at the distal end of the anchor and then pulled through the slots as it is pulled and removed out from the axial hole.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

FIG. 01: Isometric view of Right hand hemispherical deployer

FIG. 01A: Front view of Right hand hemispherical deployer, showing passer profile.

FIG. 02: Isometric view of Left hand hemispherical deployer

FIG. 02A: Front view of Left hand hemispherical deployer, showing passer profile.

FIG. 03: Isometric view of Right hand helical deployer

FIG. 03A: Front view of Right hand helical deployer, showing passer profile.

FIG. 04: Isometric view of Left hand helical deployer

FIG. 04A: Front view of Left hand helical deployer, showing passer profile.

FIG. 05: Passer tip details for all types of deployer

FIG. 06: Complete short sling

FIG. 07: Isometric view of attachment

FIG. 07A: Cross sectional view of attachment

FIG. 08: Isometric view of ring

FIG. 08A: Cross sectional view of ring

FIG. 09, 09A: Isometric view of bearing of short sling

FIG. 09B: cross sectional view of bearing of short sling

FIG. 10: Isometric view of anchor of short sling

FIG. 10A: cross sectional view of anchor of short sling

FIG. 11: Details of mesh

FIG. 12: Side view of anchor and ring

FIG. 12A: Cross sectional view of anchor and ring

FIG. 12B: Cross sectional view of assembled anchor and ring

FIG. 13: Side view of attachment assembly and bearing

FIG. 13A: Cross sectional view of attachment assembly and bearing

FIG. 13B: Cross sectional view of assembled attachment assembly and bearing

FIG. 14: Cross sectional view of complete short sling

FIG. 15A: Shows assembled short sling with the pair of hemispherical deployer

FIG. 15B: Shows assembled short sling with the pair of helical deployer

FIG. 16: Shows cutting and removing the one end of the string and bearing attachment assembly.

FIG. 17: Cross sectional view of anchor mesh assembly with string.

FIG. 18-20: Shows the procedure for inside out approach of the short sling with the delivery device.

FIG. 21-23: FIG. 35-37: Shows the procedure for inside out approach of the short sling with the delivery device.

FIG. 24-25: Placement of the short sling during surgery

FIG. 26: Shows the position of anchor after it is positioned on the obturator muscle.

FIG. 27: Complete long sling

FIG. 28: Isometric view of bearing of long sling

FIG. 28A: cross sectional view of bearing of long sling

FIG. 29: Isometric view of sheath

FIG. 30: Shows cross sectional view of bearing assembled with mesh and sheath, also assembled attachment and ring

FIG. 30A: Cross sectional view of bearing with sheath and mesh into attachment-ring assembly

FIG. 31: shows assembled long sling with pair of hemispherical handle

FIG. 32A: Shows insertion of passer in to the bearing attachment assembly from proximal end

FIG. 32B: Shows assembled passer with bearing attachment assembly from proximal end

FIG. 32C: Shows cross sectional view of assembled passer with bearing attachment assembly

FIG. 33: shows assembled long sling with pair of helical handle

FIG. 34A: Shows insertion of passer in to the bearing attachment assembly from distal end

FIG. 34B: Shows assembled passer with bearing attachment assembly from distal end

FIG. 34C: Shows cross sectional view of assembled passer with bearing attachment assembly

FIG. 35-37: Shows the procedure for inside out approach of the long sling with the delivery device.

FIG. 38-40: Shows the procedure for outside in approach of the long sling with the delivery device.

FIG. 41-45: Shows the placement, pulling, cutting and removal of sheath in a long sling approach.

### BRIEF DESCRIPTION OF EXAMPLE EMBODIMENTS

FIG. 1 illustrates 101 which is the right hand hemispherical delivery device where 102 is the handle. Referring to FIG. 1 the right hand hemispherical delivery device has a finger grip 103 and thumb grip 104. The illustration 106 in FIG. 1A is the right hand hemispherical passer of the right hand hemispherical delivery device 101. The right hand hemispherical passer 106 is used to insert the sling system in the left hand side of the subject in an inside-out approach. Referring to

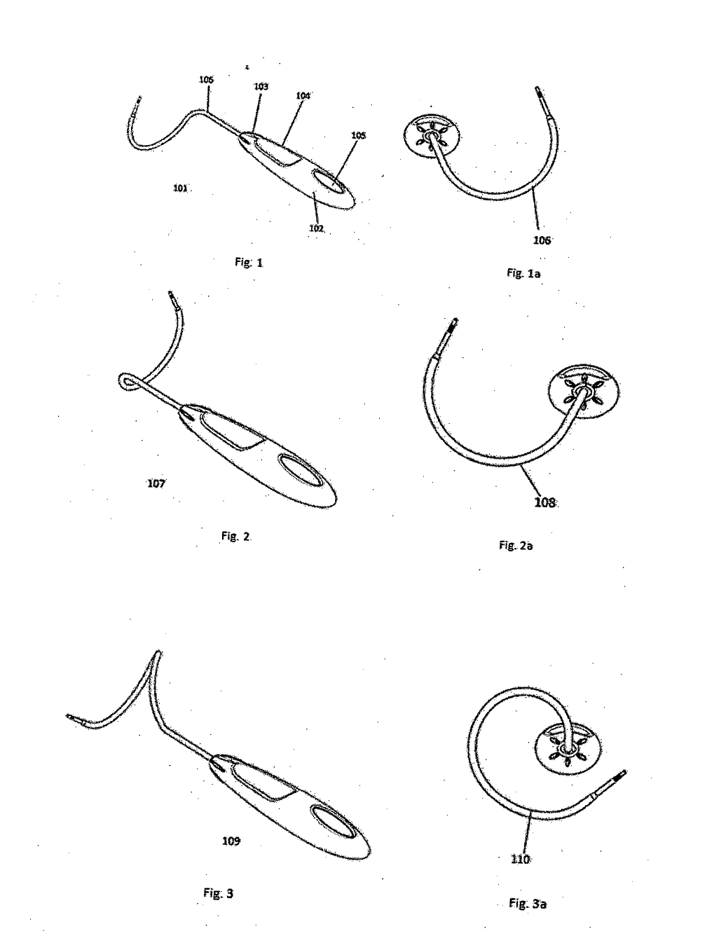

FIG. 2 illustration 107 depicts left hand hemispherical delivery device. FIG. 2A shows the illustration 108 where it depicts left hand hemispherical passer of the left hand hemispherical delivery device 107. The left hand hemispherical passer 108 is used to insert the sling system in the right hand side of the subject in an inside-out approach.

FIG. 3 illustration 109 depicts right hand helical delivery device and FIG. 3A illustration 110 depicts right hand helical passer of the right hand helical delivery device 109. The right hand helical passer 110 is used to insert the sling system in the left hand side of the subject in an outside-in approach. FIG. 4 illustrates 111 where it depicts left hand helical delivery device and FIG. 4A illustrates 112 where it depicts left hand helical passer of left hand helical delivery device. The left hand helical passer 112 is used to insert the sling system in the right hand side of the subject in an outside-in approach. FIG. 5 illustrates various features of the passer end in which 113 is the passer rod with diameter 3.15 mm and 114 is the passer rod with diameter 1.8 mm. The passer end has a helical screwing thread 115. The passer end illustrates 116 with passer rod which has a diameter of 1.4 mm. The illustration 117 in FIG. 5 shows the tip of the passer that is present in the handle. The passer end as shown in FIG. 5 and the passer tip 117 allows smooth and easy penetration of the passer in the tissues during sling insertion procedure.

FIG. 6 shows the entire short sling system 201. FIG. 7 illustrates the attachment 202 of the short sling where 203 is the distal end of attachment and 204 is the proximal end of the attachment. FIG. 7A is the cross sectional view of the attachment where 205 is the distal entry for the passer or open space at the distal end. 206 is the tapered surface. 207 is the internal helical threads that are present in the inner region of the attachment. 208 represent the inner space where the ring and bearing will be placed and fixed by various mechanical securing methods explained in further description. 209 is the base end of the attachment at the proximal end.

FIG. 8 illustrates 211 as the ring which is incorporated into the inner space 208 of the attachment 202 of the short sling system 201. The ring 211 is secured mechanically, or by other securing means to the attachment 202. In FIG. 8A the base of the ring is 212. 214 is the outer surface of the ring and 213 is the inner tapered surface. Together 212 and 214 act to cause fixation of the ring to the attachment through which the bearing will be snap fitted as will be explained in further illustrations.

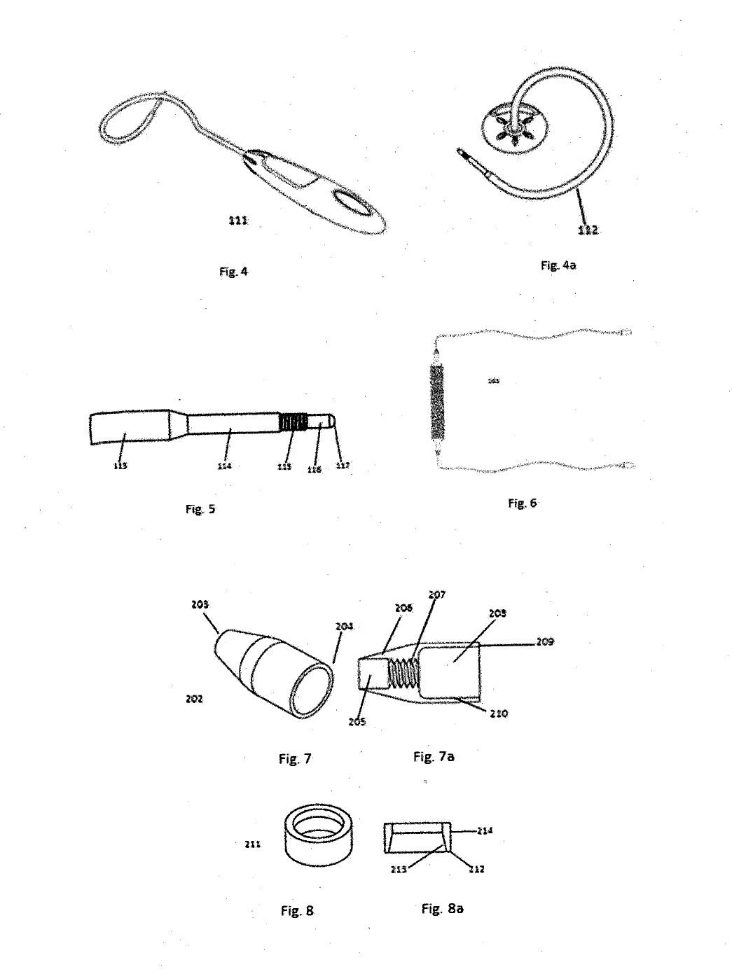

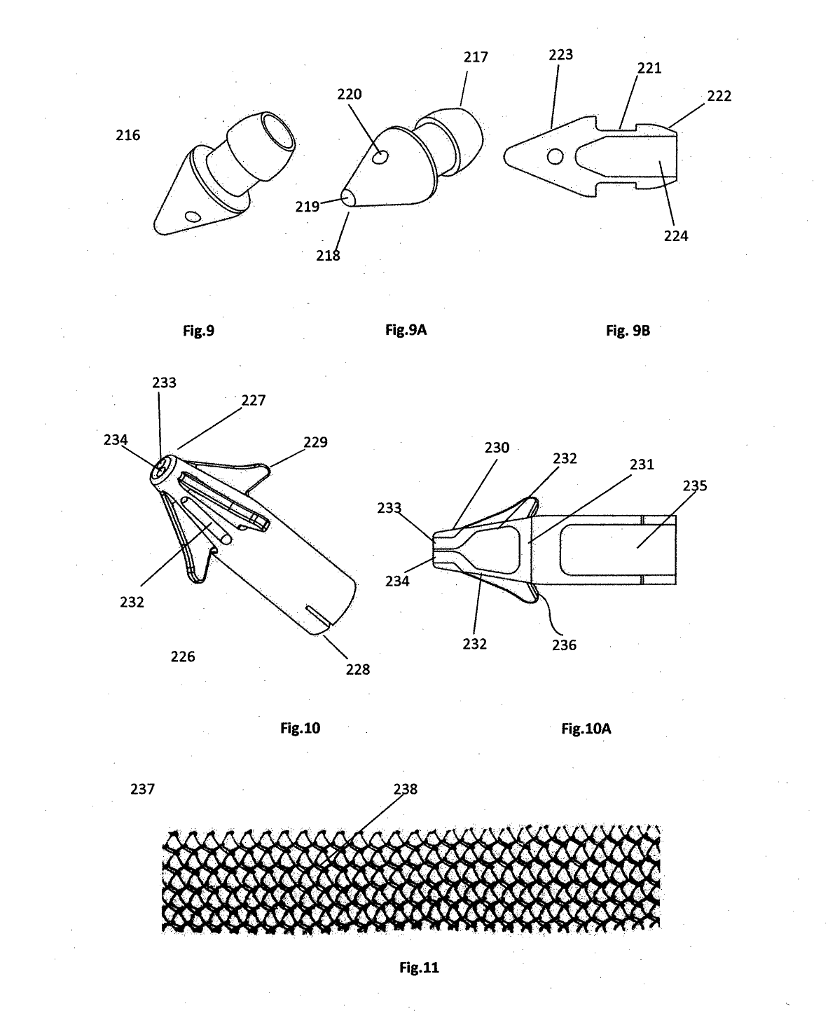

FIG. 9 depicts the bearing 216 that is incorporated to attach and fix inside the ring 211 and fixed in the inner space 208 of the attachment 202. 217 is the proximal end of the bearing and 218 is the distal end of the bearing. 219 is the tip of the bearing that allows smooth penetration in the tissues and 220 is the transverse hole through which the string will pass. 221 is the groove of the bearing wherein the groove is snap fixed to the base 212 and outer surface 214 of the ring. 222 is the tapered surface of the bearing at proximal end. This allows bearing to slide through the inner tapered surface 213 of the ring for snap fitting. 223 is the tapered surface of the bearing at distal end 218 that allows for smooth penetration of the bearing in the tissues during insertion. The bearing consists of inner hollow region 224 at the proximal end 217 of the bearing.

The ring 211 is snap fitted over the groove 221 of the bearing 216. This prevents axial displacement or dislodging of the bearing 216 from the attachment 202. Since the ring 211 and groove 221 of the bearing are cylindrical, the bearing 216 can have relative rotational motion with respect to the ring 211 and the attachment 202 but cannot have axial displacement of the bearing 216 with respect to the attachment 202 thus preventing twisting of the sling system. For attaching the sling 201 to the delivery device passer as explained above the surgeon needs to rotate the attachment 202 for fixation by threading 207 into the distal entry or open space 205 of the attachment 202 with the threading 115 of the passer end, since attachment can have relative rotational motion with respect to the bearing it can prevent the bearing and the sling to get twisted.

FIG. 10 depicts the anchor 226 of the short sling system 201. The distal end of the anchor 227 consists of multiple number of fins 229 which is present so that the fins are fixed to the tissues of the obturator foramen for proper placement of the short sling system 201. On the distal end of anchor 227 the anchor consists of two axial holes 233 and 234. Internal cavity 235 as shown in FIG. 10A on the proximal end 228 of anchor 226 is present for insertion of mesh 237. The mesh as shown in FIG. 11 will be placed beneath the urethra for support. 230 is the tapered surface of the bearing. 232 is the slot on the tapered surface.

The string is inserted in the axial hole 233 at the distal end 227 of the anchor. The string is passed in the slot 232 at the tapered surface 230, then through the slot 231 and then passed through the slot 232 and removed out from the axial hole 234. The string is knotted (knot shown as 242) at the end after passing through the internal passage and later the knot is placed inside the transverse hole 220 of the bearing 216 and is welded or joined or glued as shown in FIG. 14. The knot 242 won't expose outside during tissue retraction or surgery which is an advantage over other inventions.

FIG. 12, 12A, 12B display the fixation 240 of the attachment 202 and the ring 211 by various securing means such as mechanical, any other securing means etc. FIG. 12 displays the outline shape of the attachment 202 and the ring 211. FIG. 12A displays the cross sectional view of the attachment 202 and the ring 211 where the attachment is fixed to the ring in a manner as shown in FIG. 12B where the base 212 and outer surface 214 act to cause fixation of the ring 211 to the base 219 and outer surface 210 of the attachment 202 when the ring 211 is inserted into the inner space 208 of the attachment.

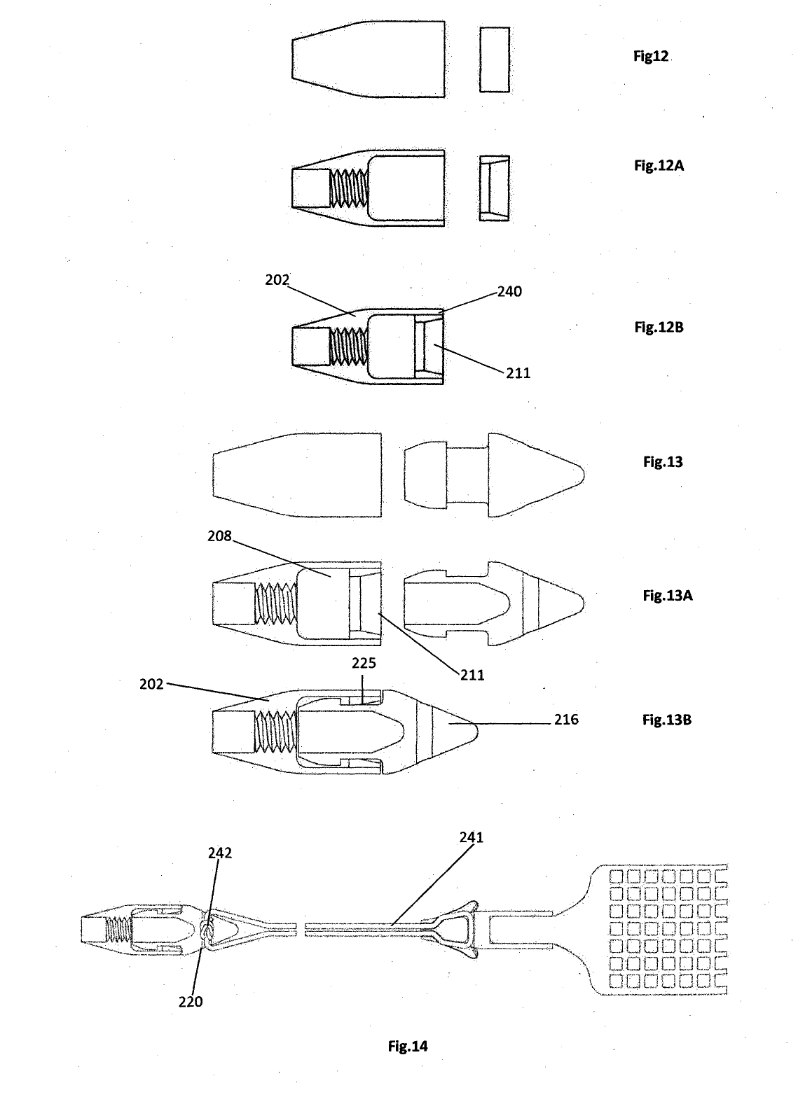

FIG. 13 shows the cross sectional outline view of the attachment 202 and the bearing 216 that will be fixed together. FIG. 13A represents a bearing 216 that is incorporated to attach and fix inside the ring 211 and fixed in the inner space 208 of the attachment 202. The ring 211 gets fixed through snap fitting 225 as shown in FIG. 13B inside the groove 221 of the bearing 216. 222 is the tapered surface of the bearing 216 as shown in FIG. 9B that allows the bearing 216 to slide through the inner tapered surface 213 as shown in FIG. 8A of the ring 211 for snap fitting.

FIG. 14 shows a view where the mesh 237 is joined to the anchor 226 at the proximal end 228 of anchor 226 wherein the anchor 226 is further joined with the string 241 at the distal end 228.

The string 241 is looped through the internal cavity and through the transverse hole 220 of the bearing and knotted 242 at the end. Later the knot is placed inside the transverse hole 220 of the bearing 216 and is welded or joined or glued. This prevents the knot 242 to be exposed outside as shown in FIG. 14 during tissue retraction or surgery as explained above.

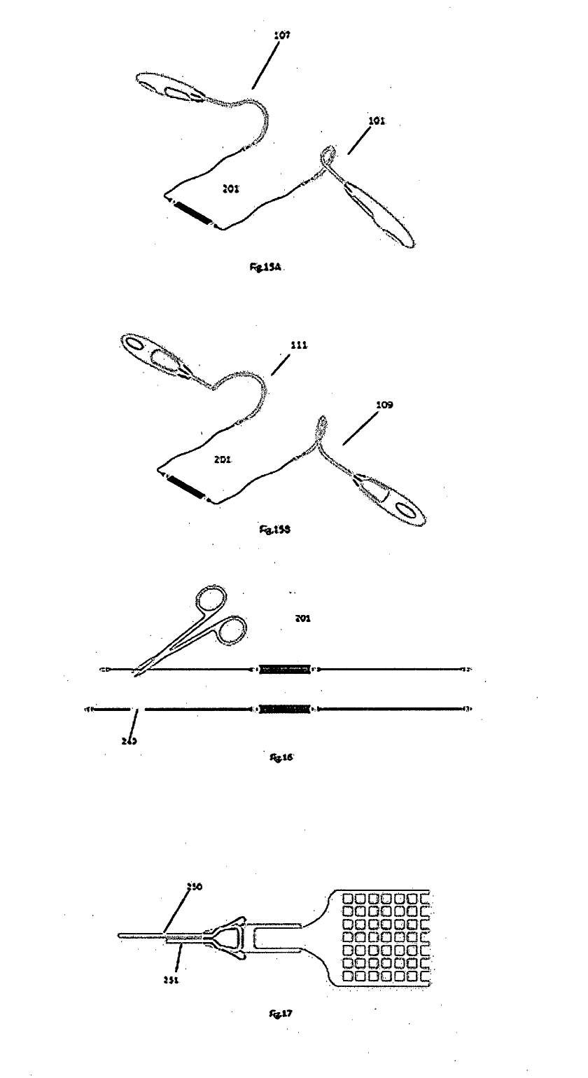

FIG. 15A shows the assembly of short sling system 201 with right hand hemispherical delivery device 101 and left hand hemispherical delivery device 107 for inside-out approach.

FIG. 15B shows the assembly of short sling system 201 with right hand helical delivery device 109 and left hand helical delivery device 111 for outside-in approach.

FIG. 16 shows the cutting of the exposed string 241 strips to allow removal of the string 241 from the anchor 226 by pulling the free end of the string 241 to remove the entire string 241 from the body, the string from side 250 is pulled and string from side 251 is retracted from the anchor 226 as shown in FIG. 17. The string 241 is pulled through the axial hole 233 at the distal end 227 of the anchor and then pulled through the slot 232 at the tapered surface 230, then through the slot 231 and then it is passed through the slot 232 as it is pulled and removed out from the axial hole 234.

The present invention is designed to prevent the leakage of urine caused by incontinence, which may result from an increase in intra-abdominal pressure due to activities such as coughing, laughing, sneezing and exercising or, alternatively, can be caused by weakened pelvic floor muscles, a weakened external sphincter, a urethra which has lost muscle tone, or an abnormally short urethra. The present invention is also designed to prevent the leakage of urine caused by urge or mixed incontinence.

Once the procedure of insertion of the short sling system 201 is complete from both the sides, the string is pulled so that the anchors 226 are tensioned in the tissues of the obturator foramen. This allows fixation of the anchors and proper placement of mesh 237 beneath the urethra. Mesh is shown in FIG. 11. Cut the exposed string 241 strips as shown in FIG. 16. The free end of the string 241 after cutting shown as 249 is pulled to remove the entire string 241 from the body, the string from side 250 is pulled and string from side 251 is retracted from the anchor 226 as shown in FIG. 17.

The assembly for insertion of short sling system 201 in an inside-out approach as shown in FIG. 15A involves connecting the attachment 202 of left hand hemispherical delivery device 107 as shown in FIG. 2 to one end of the string 241 by threading. Rotating the threading 207 of the attachment 202 for three times. The threading attachment prevents relative rotational movement of the mesh during and after insertion of the sling system.

The procedure involves placing the right hand hemispherical passer 106 as shown in FIG. 1A of the right hand hemispherical delivery device 101 at the vaginal incision at the right hand side of the patient and at right hand side of the surgeon as shown in FIG. 18 and push the right hand hemispherical passer 106 through the tissue so that the passer tip 117 traverses the tissues of obturator foramen.

The device is inserted slowly inward, traversing and slightly passing into the tissues of the obturator foramen. While inserting the short sling system 201, it is made sure that the handle 102 of the right hand hemispherical delivery device 101 is oriented so that the straight tip of the right hand hemispherical passer 106 is aligned and remains in the straight orientation until the tip of the right hand hemispherical passer 106 traverses the obturator foramen.

There are anchors 226 present on both the sides of the short sling support system called the mesh 237. The distal end of the anchor is connected to the string 241 as explained in above FIG. 10 and FIG. 10A. The anchors 226 present on both the sides of mesh 237 of the short sling support system pierces the tissues of the obturator foramen thus anchoring the fins 229 of the anchor into the tissues of the foramen. The anchor fins 229 gets firmly placed in the tissues of the obturator foramen as shown in FIG. 26. The handle 102 is moved towards the midline until the handle is vertical to the floor.

The point of the right hand hemispherical passer 106 should exit near the previously determined exit point at the level of the clitoris in the fold of the thigh as shown in FIG. 19. Slight skin manipulation may be required. Make an incision at the point where the tip of the passer protrudes through the skin.

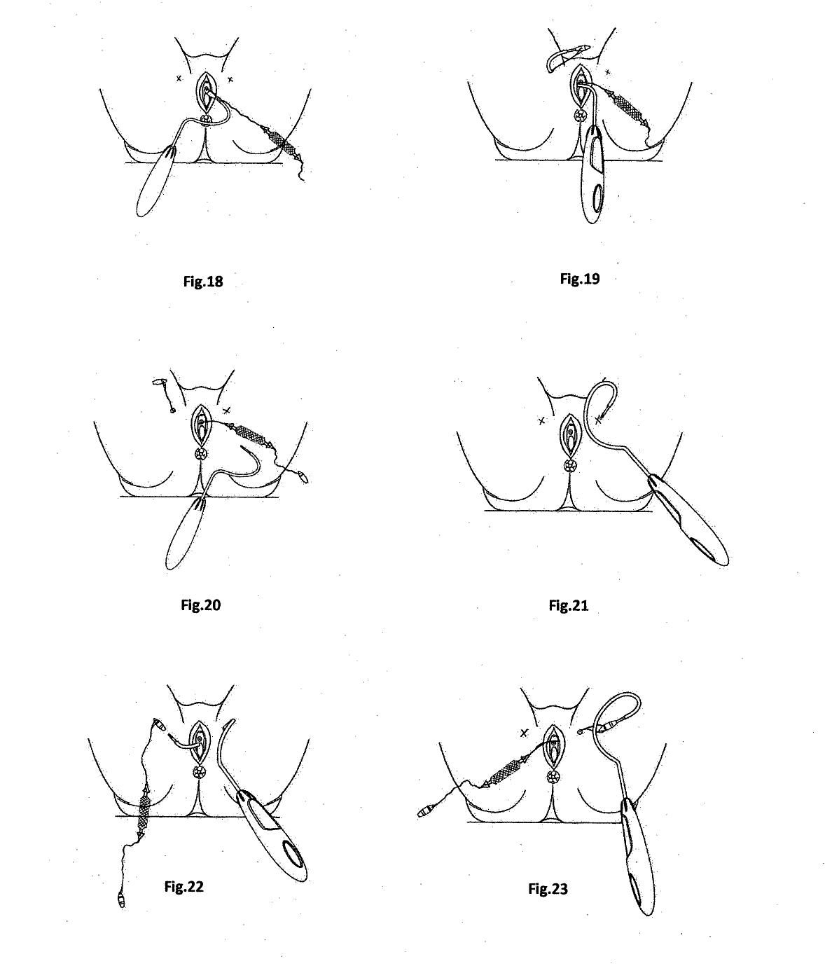

When the attachment along with some portion of string 241 appears at the skin opening, grasp it with a forcep. Now detach the attachment 202 from the right hand hemispherical delivery device 101 and retrieve the passer by reverse rotation of the handle 102 as shown in FIG. 20.

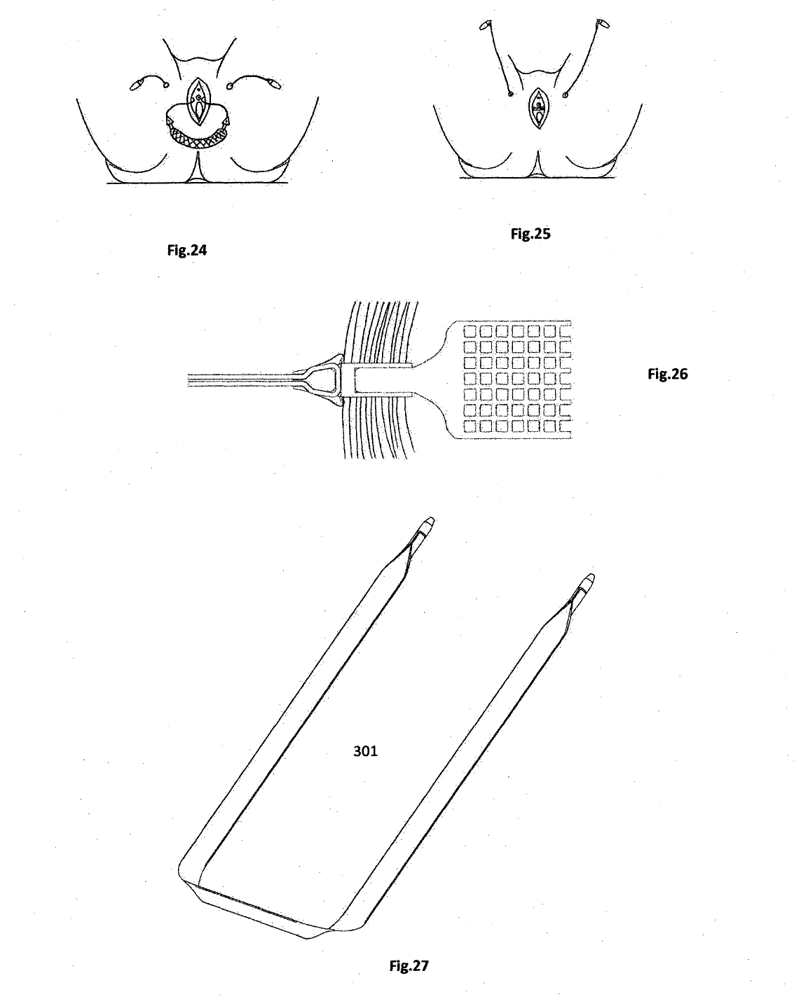

Repeat the technique on the patient's left hand side with the left hand hemispherical delivery device 107 as shown in FIG. 1. Once the procedure of insertion of the short sling system 201 is complete on left hand side of the patient, detach the attachment 202 from the left hand hemispherical passer 108 of the left hand hemispherical delivery device 107 as shown in the procedure FIG. 24, then pull both the free end of the string 241 from both sides as illustrated in FIG. 25 to position the mesh 237. Pull it until the anchor 226 on the ends of the mesh 237 gets fixed to tissues of the obturator foramen as shown in FIG. 26. Cut the exposed string strips as shown in FIG. 16. The free end of the string after cutting as shown in 249 is pulled to remove the entire string from the body, the string from side 250 is pulled and string from side 251 is retracted as shown in FIG. 17. Close each skin incision. Close the vaginal incision.

Once the anchors 226 are tensioned in the tissues of the obturator foramen the free end of the string 241 is pulled out which emerges out of the incision and can be removed so as to reduce discomfort in a patient. No string remains inside the body after the procedure of insertion is complete.

The assembly as shown in FIG. 15B for inserting the short sling system 201 for supporting the urethra in an outside-in approach involves the procedure of using right hand helical delivery device 109 as shown in FIG. 3, involves making an incision at one of the previously marked position at the level of clitoris in the folds of the thigh as shown in FIG. 21 at the left hand side of the patient. Insert the right hand helical passer 110 through this incision and push it to perforate the tissues of the obturator foramen.

Keep the handle 102 close to the patient and rotate the handle 102 to direct the handle passer tip 117 towards the urethra. Place the index finger of the opposite hand on the same side to ensure that the passer does not perforate the vagina or go above the vaginal dissection.

Guide the passer to meet the index finger. In case it does not, gently retract the right hand helical passer 110 as shown in FIG. 3A of the right hand helical delivery device 109 as shown in FIG. 3 and slowly advance until it can be located. The point of the handle 102 should exit near the previously determined exit point at the vaginal epithelium beneath the level of mid-urethra as shown in FIG. 22. Slight skin manipulation may be required.

Make an incision at the point where the tip 117 of the passer which is shown in FIG. 5 of the right hand helical passer 110 which is shown in FIG. 3A of the handle 102 protrudes through the skin. When the complete right hand helical passer thread 115 as shown in FIG. 5 along with its straight edge appears at the skin opening, connect the attachment 202 illustrated in FIG. 7 on one end of the string 241 to the right hand helical passer 110 threading 115 which is shown in FIG. 5. This procedure is illustrated in FIG. 22 Rotate the threading attachment for three times. The threading attachment prevents twisting of the mesh during and after insertion of the sling system due to the features of the ring 211, attachment 202 and the bearing 216 as explained in above illustrations.

Gently apply traction on the handle to draw the passer back through the thigh incision (the string gets pulled simultaneously along with the passer, the sling follows the passer path and the end of the sling comes out through the thigh incision.) till the string can be held by forceps. When the attachment along with some portion of string appears at the skin opening as shown in FIG. 23, grasp it with a forcep. Detach the attachment 202 from the right hand helical passer 110 and retrieve the handle by reverse rotation of the handle 102 as shown in FIG. 23. Repeat the technique on the patient's right hand side. Then pull both the free end of the string 241 from both sides as explained above to position the mesh. Pull it until the anchor 226 on the end of the mesh 237 gets fixed in the tissues of obturator foramen. Cut the exposed string 226 strips and pull it to remove the entire string from the body. Close each skin incision. Close the vaginal incision.

Pulling of the string 226 will ensure proper positioning of the sling inside the tissues. The string 241 can then be removed after the attachment on the string is visible so that no string remains inside the body causing unnecessary discomfort to the subject and the tissue. There are less chances of string erosion occurring due to removal of string from the implant and the body. The pulling of the string 241 will ensure full proper placement of the sling under the urethra.

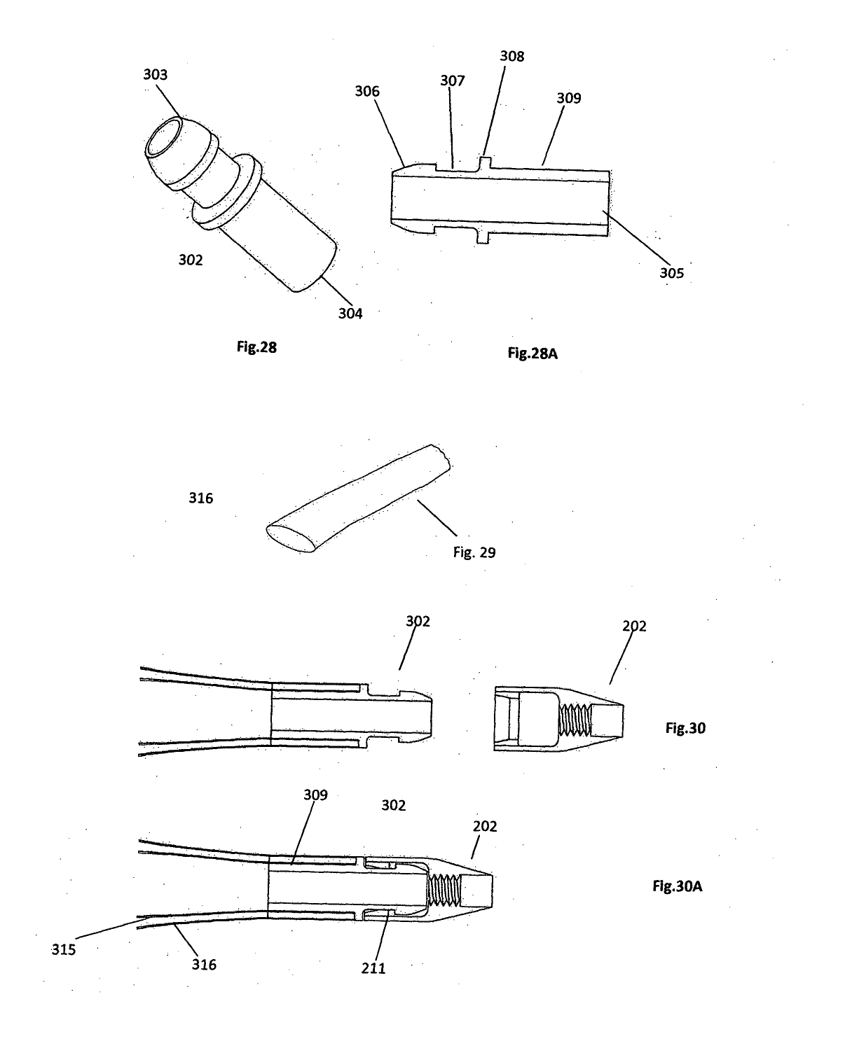

FIG. 27 represents the long sling system 301. Illustration 302 of FIG. 28 shows the bearing of the long sling system 301. FIG. 28A shows the cross sectional view of the bearing 302. The bearing 302 of the long sling system 301 has proximal end 304 and distal end 303 as shown in FIG. 28. 306 is the tapering surface at the distal end 303 of the bearing and 307 is the step or the groove at the distal end 303 of the bearing. The tapered surface of the bearing 216 will slide through the inner tapered surface 213 as shown in FIG. 8A of the ring 211 for snap fitting. The ring will be snap fitted to the groove of the bearing as explained in further illustrations.

FIG. 8 illustrates 211 as the ring which is incorporated into the inner space 208 of the attachment 202. The ring 211 is secured mechanically, welded or by other securing means fixed to the inner part of the attachment 202. In FIG. 8A the base of the ring is 212. 214 is the outer surface of the ring and 213 is the inner tapered surface. Together 212 and 214 act to cause fixation of the ring to the base 209 and outer surface 210 of the attachment through which the bearing will be snap fitted as will be explained in further illustrations.

FIG. 30 shows the bearing 302 fixed to the mesh as will be explained in further illustrations. FIG. 30 also shows fixation of ring 211 and the attachment 202. FIG. 28 depicts the bearing 302 that is incorporated to attach and fix inside the ring 211 and the fixed in the inner space 208 of the attachment 202 as shown in FIG. 30A. 307 is the groove of the bearing 302 wherein the groove is snap fixed to the base 212 and outer surface 214 of the ring. 306 is the tapered surface of the bearing at distal end 303. This allows bearing 302 to slide through the inner tapered surface 213 of the ring for snap fitting as shown in FIG. 30A. The bearing consists of inner hollow region 305 at the proximal end 304 of the bearing 302.

The ring 211 is snap fitted over the groove 307 of the bearing 302. This prevents axial displacement or dislodging of the bearing 216 from the attachment 202. Since the ring 211 and groove 221 of the bearing 302 are cylindrical, the bearing 302 can have relative rotational with respect to the ring 211 and the attachment 202 but cannot have axial displacement of the bearing 302 with respect to the attachment 202 thus preventing twisting of the sling system and the mesh 315.

The mesh 315 of the long sling system 301 has sheath 316 as shown in FIG. 29 on its outer surface. The mesh 315 having outer covering sheath 316 is fixed by gluing, snapping, mechanical or any other securing means to the outer cylindrical surface 309 of the bearing 302. The mesh with outer covering sheath 316 is placed and wrapped to the bearing 302 outer cylindrical surface 309 till the stopper or limiting factor 308 which is shown in FIG. 28A. The entire sheath 316 is pulled and retracted from the outer edges of the mesh, cutting the exposed mesh strips and pulling it to remove the exposed strip from the body.

FIG. 31 shows inside-out assembly of the long sling system with both left hand delivery device 107 and right hand delivery device 101.

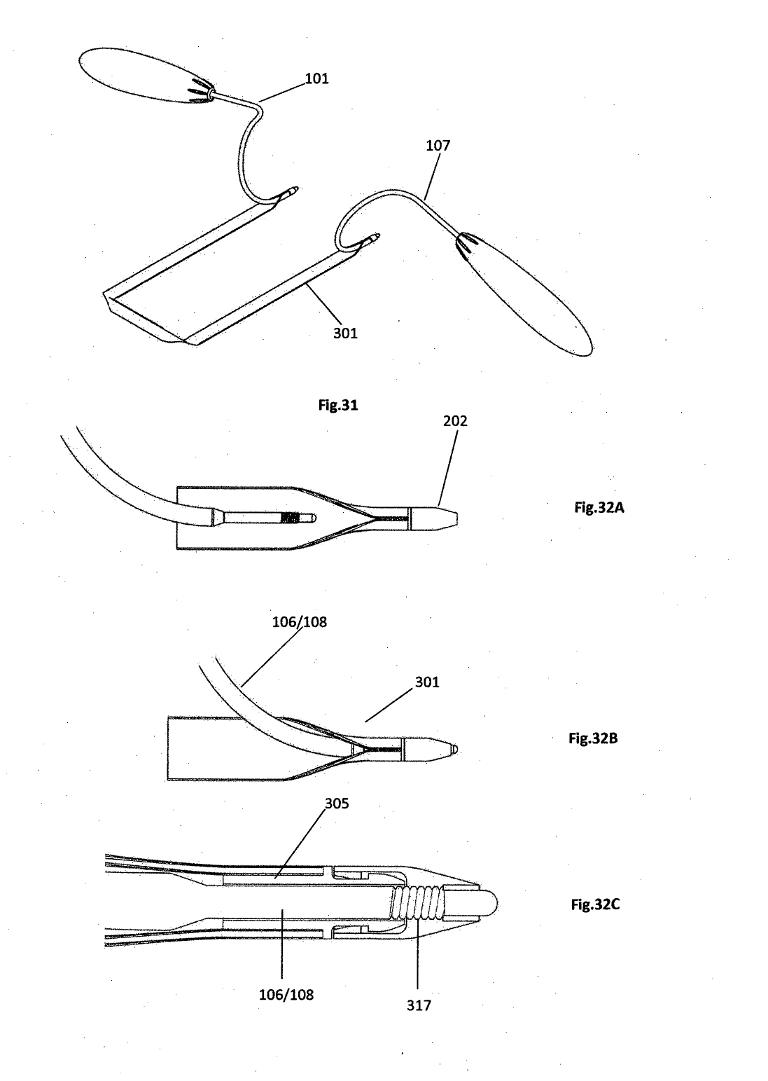

FIG. 32A, 32B and 32C display the inside-out approach of the long sling assembly. FIG. 32A and 32B display the method of insertion of the hemispherical passer into the space of the bearing and the attachment. The right hand hemispherical passer 106 or the left hand hemispherical passer 108 is inserted into the inner space 305 of the bearing 302 as shown in FIG. 32B for attachment. Then it is inserted into the inner space 208 of the attachment 202 as shown in FIG. 3C and fixed by threading 115 of the passer. Inner space 305 of the bearing 302 is as shown in FIG. 28A.

Illustration FIG. 33 displays the outside-in approach of the long sling assembly with both left hand delivery device 111 and right hand delivery device 109.

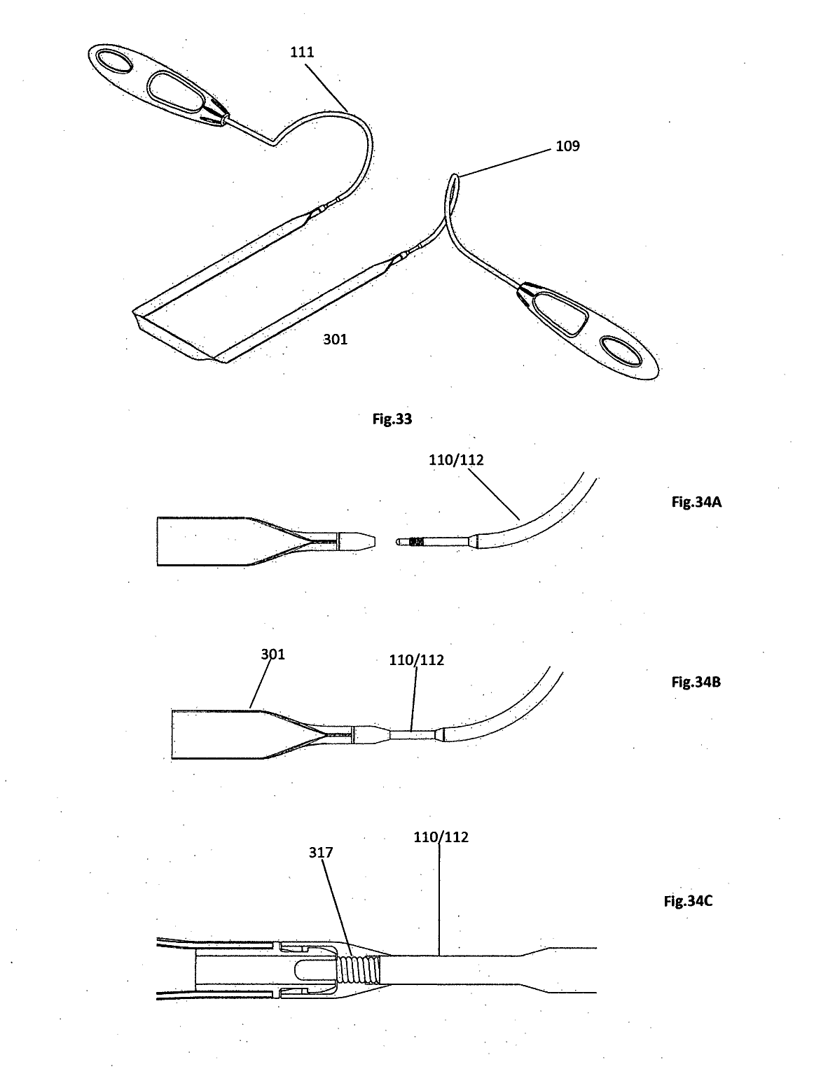

FIG. 34A, 34B and 34C display the outside-in approach of the long sling assembly. FIG. 34A and 34B display the method of insertion of the helical passer into the space of the bearing and the attachment.

The left hand helical passer 112 and right hand helical passer 110 is inserted into the inner space 208 of the attachment 202 as shown in FIG. 34A for attachment by screwing/threading. Then it is inserted into inner space 305 of the bearing as shown in FIG. 34B. The FIG. 34A, 34B and 34C illustrate the above explained embodiment. Inner space 305 of the bearing is as shown in FIG. 28A.

The procedure for the implantation of long sling system 301 in an inside-out approach as shown in FIG. 35 using right hand hemispherical delivery device 101 as shown in FIG. 1 and left hand hemispherical delivery device 107 as shown in FIG. 2 involves making an incision through the marked position at the vaginal epithelium, beneath the level of mid-urethra, as per standard procedure for a midurethral sling. Take one side delivery device (either Right hand side or Left hand side). As per FIG. 35 the right hand hemispherical delivery device 101 as shown in FIG. 1 is taken by the surgeon. Connect the attachment 202 which is shown in FIG. 7A of one end of the long mesh 315 to the right hand hemispherical delivery device 101 by threading 207 which is shown in FIG. 7A by screw joint 317 as shown in FIG. 32C. Rotate the threading attachment for three times. The threading attachment prevents twisting of the mesh during and after insertion of the sling system. The ring 211 is snap fitted over the groove 307 of the bearing 302. This prevents axial displacement or dislodging of the bearing 216 from the attachment 202. Since the ring 211 and groove 221 of the bearing 302 are cylindrical, the bearing 302 can have relative rotational with respect to the ring 211 and the attachment 202 but cannot have axial displacement of the bearing 302 with respect to the attachment 202.

As displayed in FIG. 35 place the right hand hemispherical passer 106 at the surgeon's right hand side as shown in FIG. 3A at the vaginal incision at the right hand side of the patient and push the passer tip 117 through the tissue so that the tip passes the tissues of the obturator foramen.

The point of the passer tip 117 should exit near the previously determined exit point at the level of the clitoris in the fold of the thigh as shown in FIG. 36. Slight skin manipulation may be required. Make an incision at the point where the tip of the passer protrudes through the skin.

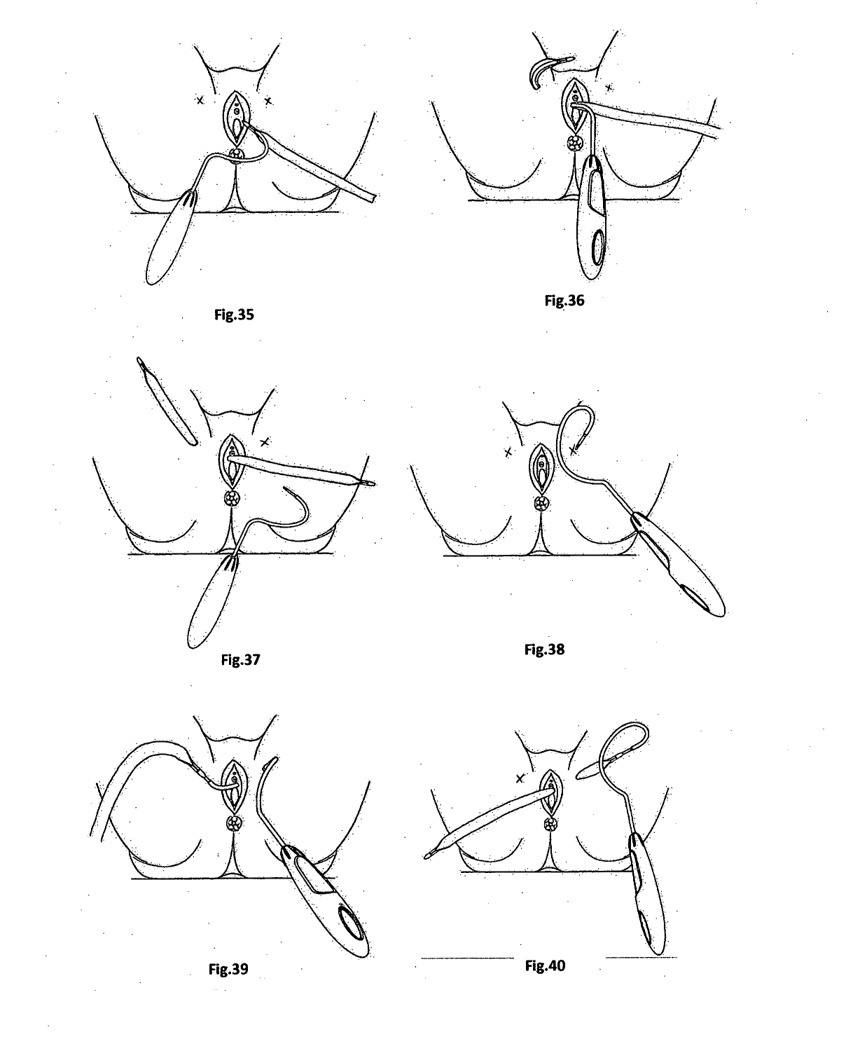

The attachment 202 is detached and the handle 102 is retrieved as shown in FIG. 37 after the procedure of insertion on one side is complete. When the attachment appears at the skin opening, grasp it with a forcep. Now detach the attachment 202 from the threading 207 which is shown in FIG. 7A and from the threading 115 which is shown in FIG. 5 of the right hand hemispherical passer 106 which is shown in FIG. 1A. Then retrieve the handle 102 by reverse rotation of the handle 102. Repeat the technique on the patient's left hand side with left hand hemispherical delivery device 107 which is shown in FIG. 2. Then pull both the free end of the mesh 315 from both sides to position it. After positioning cut and remove the sheath 316 by pulling its visible portion. Cut the exposed mesh strips at the level of the subcutaneous tissue. Close each skin incision. Close the vaginal incision.

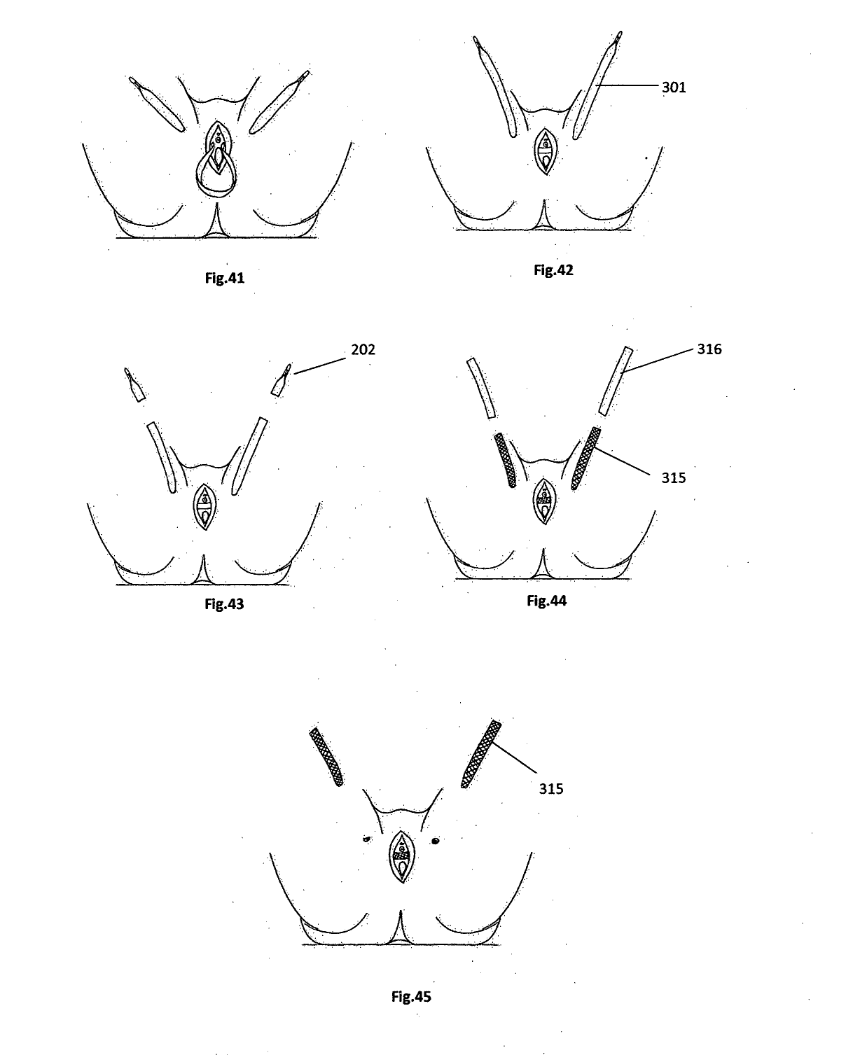

After the insertion procedure of the long mesh 301 is complete from both the sides as shown in FIG. 41. The visible free end of the mesh is pulled from both the sides as shown in FIG. 42. The attachment assembly is cut as shown in FIG. 43. The sheath is pulled from both the ends as shown in FIG. 44. The exposed mesh strips are cut at the level of the subcutaneous tissue as shown in FIG. 45 and each skin incision is closed.

The procedure for placement of long sling system 301 in an outside-in approach involves making an incision at one of the previously marked position at the level of clitoris in the folds of the thigh. Insert the tip 117 of the right hand helical passer 110 which is shown in FIG. 3A of the right hand helical delivery device 109 through this incision at the left hand side of the patient as shown in FIG. 38 and push it to perforate the tissues of the obturator membrane.

Keep the right hand delivery device 109 close to the patient and rotate the handle 102 to direct the right hand helical passer tip 117 of the right hand helical passer 110 which is shown in FIG. 3A of the right hand helical delivery device 109 which is shown in FIG. 3 towards the urethra. Place the index finger of the opposite hand on the same side of the patient to ensure that the right hand helical passer tip 117 does not perforate the vagina or go above the vaginal dissection. Guide the passer to meet the index finger. In case it does not, gently retract the right hand helical passer 110 as shown in FIG. 3A and slowly advance until it can be located. The point of the passer tip 117 should exit near the previously determined exit point at the vaginal epithelium beneath the level of mid-urethra as shown in FIG. 39.

Slight skin manipulation may be required. Make an incision at the point where the tip 117 of the right hand helical passer 110 protrudes through the skin. When the complete passer thread 115 along with its straight edge appears at the skin opening at vagina, connect the threading 207 of the attachment 202 which is shown in FIG. 7A of one end of the long mesh 301 to the passer threading 115 which is shown in FIG. 5 as shown in FIG. 39. Rotate the threading attachment for three times. The threading attachment prevents twisting movement of the mesh during and after insertion of the sling system.

Gently apply traction on the handle to draw the passer back through the thigh incision (the mesh 315 gets pulled simultaneously along with the passer, The sling follows the passer path and the end of the sling comes out through the thigh incision.) as shown in FIG. 40. The threading attachment prevents twisting movement of the mesh during and after insertion of the sling system. The ring 211 is snap fitted over the groove 307 of the bearing 302. This prevents axial displacement or dislodging of the bearing 216 from the attachment 202. Since the ring 211 and groove 221 of the bearing 302 are cylindrical, the bearing 302 can have relative rotational motion with respect to the ring 211 and the attachment 202 but cannot have axial displacement of the bearing 302 with respect to the attachment 202. The sling follows the passer path and the end of the sling comes out through the thigh incision.

When the attachment 202 appears at the skin opening near the thigh region, grasp it with forceps. Now detach the attachment 202 from the right hand helical passer 110. Then retrieve the passer by reverse rotation of the handle.

Repeat the technique on the patient's right hand side with the left hand helical delivery device 109 which is shown in FIG. 3. Take care to ensure that the sheath assembly is not twisted during the entire procedure. Then pull both the free end of the mesh 315 from both sides to position it. After positioning cut and , remove the sheath 316 by pulling its visible portion as shown in FIG. 43 and FIG. 44. Cut the exposed mesh strips at the level of the subcutaneous tissue and close each skin incision. Close the vaginal incision.

After the insertion procedure of the long mesh 301 is complete from both the sides as shown in FIG. 41. The visible free end of the mesh is pulled from both the sides as shown in FIG. 42. The attachment assembly is cut as shown in FIG. 43. The sheath is pulled from both the ends as shown in FIG. 44. The exposed mesh strips are cut at the level of the subcutaneous tissue as shown in FIG. 45 and each skin incision is closed.

We claim:

1. A device and a method for the treatment of incontinence in female patients:
comprising of a short sling system that allows removal of the string from the internal passage of the anchor assembled on both the sides of the mesh, the short and long sling system comprising anti-twist element that prevents anti-twisting of the sling system during and after insertion and during assembly with the delivery device, and a method that allows inside-out and outside-in approach of the short and long sling system using right hand hemispherical delivery device, left hand hemispherical delivery device, right hand helical delivery device, left hand helical delivery device.

2. The device as claimed in claim 1, wherein the removal of string comprises pulling the string from the end that is joined to the distal end of the bearing and the proximal end of the attachment after cutting the exposed string strips and subsequent retraction of the string from the body at other end, with the other end of the string joined to distal end of the anchor.

3. The device as claimed in claim 1, wherein the removal string is passed through the internal passage of the slots present in the anchor through the two axial holes contained in the anchor.

4. The device as claimed in claim 1, wherein the short sling and long sling anti-twist element comprises fixation of the bearing in the inner space of the attachment and the ring on both the ends of the sling system, wherein the base and the outer surface of the ring is fixed to the outer surface and the base of the attachment, where the outer surface and base of the ring is snap fitted on the groove of the bearing.

5. The device as claimed in claim 1, wherein the attachment of the sling system with the delivery device is achieved through a mechanism that allows rotation of the threading of the attachment with the passer and to retrieve the passer by reverse rotation of the handle.

6. The device as claimed in claim 1, wherein the anti-twist element facilitates relative rotational movement of the bearing with the attachment and the ring and prevents axial displacement of the bearing with respect to the attachment during and after insertion.

7. The method for the treatment of incontinence in female patients as claimed in claim 1, using right and left hand hemispherical delivery device, comprising attaching the short sling system in an inside-out approach to the passer threading of delivery device, making a vaginal incision, placing the sling beneath the mid-urethra through transobturator approach, allowing the fins of the anchor to tension in the tissues of the obturator foramen within the pubic bones, pulling the string until the anchor on the mesh end gets fixed to obturator muscles, the anti-twist element prevents twisting of the mesh, pulling and retracting the entire string system from the distal end of the anchor, cutting the exposed string strips and pulling and retracting it to remove the entire strip from the body. Closing the incisions.

8. The method for the treatment of incontinence in female patients as claimed in claim 1 using right and left hand helical delivery device, comprising attaching the short sling system in an outside-in approach to the passer threading of the delivery device, making a thigh incision at the marked position, allowing the point of the handle to exit near the previously determined exit point at the vaginal epithelium beneath the level of mid-urethra, connecting the attachment on one end of the string to the passer threading, rotating the threading, gently applying traction on the handle to draw the passer back through the thigh incision, till the string can be held by forceps, repeating the procedure on the other side of the patient, allowing the fins of the anchor to tension in the tissues of the obturator foramen within the pubic bones, pulling the string until the anchor on the mesh end gets fixed to obturator muscles, placing the sling beneath the mid-urethra through transobturator approach, the anti-twist element prevents twisting of the mesh, pulling and retracting the entire string system from the distal end of the anchor, cutting the exposed string strips and pulling and retracting it to remove the entire strip from the body. Closing the incisions.

9. The method for the treatment of incontinence in female patients as claimed in claim 1 using right and left hand hemispherical delivery device, comprising attaching the long sling system in an inside-out approach to the passer threading of the delivery device, making a vaginal incision, placing the sling beneath the mid-urethra through transobturator approach, allowing the fins of the anchor to tension in the tissues of the obturator foramen within the pubic bones, the anti-twist element prevents twisting of the mesh, pulling the free ends of the mesh, cutting the attachment on the mesh ends, pulling and positioning the free ends of the mesh, pulling and retracting the entire sheath from the outer edges of the mesh, cutting the exposed mesh strips and pulling it to remove the exposed strip from the body. Closing the incisions.

10. The method for the treatment of incontinence in female patients as claimed in claim 1 using right and left hand helical delivery device, comprising attaching the long sling system in an outside-in approach to the passer threading of the delivery device, making a thigh incision at the marked position, allowing the point of the handle to exit near the previously determined exit point at the vaginal epithelium beneath the level of mid-urethra, connecting the attachment on one end of the mesh to the passer threading, rotating the threading, gently applying traction on the handle to draw the passer back through the thigh incision, till the sling can be held by forceps, repeating the procedure on the other side of the patient, allowing the fins of the anchor to tension in the tissues of the obturator foramen within the pubic bones, pulling the sling until the anchor on the mesh end gets fixed to obturator muscles, placing the sling beneath the mid-urethra through transobturator approach, the anti-twist element prevents twisting of the mesh, cutting the attachment on the mesh ends, pulling and positioning the free ends of the mesh, pulling and retracting the entire sheath from the outer edges of the mesh, cutting the exposed mesh strips and pulling it to remove the exposed strip from the body. Closing the incisions.
